(19) United States
(12) Patent Application Publication
(10) Pub. No.: US 2008/0145853 A1
(43) Pub. Date: Jun. 19, 2008

(54) Methods and Kits for Detection of Chromosome Aneuploidy by High Performance Liquid Chromatography with Post-Column Fluorescence Detection

(76) Inventors: Grant Wu, Omaha, NE (US); Benjamin L. Legendre, Omaha, NE (US); Jim Jianzhong Zhu, Omaha, NE (US)

Correspondence Address: LICATA & TYRRELL P.C., 66 E. MAIN STREET, MARLTON, NJ, 08053, US

(21) Appl. No.: 11/909,840

(22) PCT Filed: Mar. 28, 2006
(86) PCT No.: PCT/US2006/011494

§ 371 Date: Oct. 17, 2007

Related U.S. Application Data
(60) Provisional application No. 60/666,246, filed on Mar. 29, 2005.

(51) Int. Cl. C12Q 1/68

(52) U.S. Cl. 435/6

(57) ABSTRACT

Methods and kits for determining aneuploidy of selected chromosomes by multiplex polymerase chain reaction (PCR) and post-column fluorescence high performance liquid chromatography (PCF-HPLC) are provided.

This patent application claims the benefit of priority from U.S. Provisional Application Ser. No. 60/666,246, filed Mar. 29, 2005, teachings of which are herein incorporated by reference in their entirety.

### FIELD OF THE INVENTION

The present invention provides methods and kits for determining aneuploidy of selected chromosomes by multiplex polymerase chain reaction (PCR) and post column fluorescence-high performance liquid chromatography (PCF-HPLC).

### BACKGROUND OF THE INVENTION

Chromosomal aneuploidy is the cause of a number of genetic diseases and disorders. Such disorders include Patau syndrome (trisomy of chromosome 13), Edward syndrome (trisomy 18), Down syndrome (trisomy 21), Turner syndrome (absence of an X sex chromosome-XO), and Kleinfelter syndrome associated with extra X or Y sex chromosomes (e.g. XXY, XYY, XXX, etc.). Since approximately 1 in 200 live human births are estimated to have numerical chromosomal abnormalities (see marchofdimes with the extension .com/professionals/681_{—}1209.asp of the world wide web), an accurate, inexpensive, and rapid diagnosis is needed, particularly for pre-natal screening.

A variety of techniques exist for detecting the presence of aneuploidy, including karyotype analysis by microscopy (Ricciardiello et al. Cancer Research 2003 63(21):7256-62), interphase fluorescence in-situ hybridization (FISH, Lue et al. Endocrinology 2001 142(4):1461-70), quantitative fluorescence PCR (QF-PCR, Goddijn et al. Gynecologic and Obstetric Investigation 2005 60(3):139-44), comparative genomic hybridization (CGH, Morrison et al. Journal of Clinical Oncology 2005 223(36):9369-76), and Multiplex Ligation-dependent Probe Amplification (MLPA, Slater et al. Journal of Medical Genetics 2003 40(12):907-12). Unfortunately, many of these approaches are either technically difficult to perform (requiring highly skilled personnel), expensive, require specially-designed fluorescent probes, or rely on the subjective evaluation of a cytogeneticist which can lead to misdiagnosis. Recently, multiplex PCR/liquid chromatography has been used to determine exon copy number anomalies due to gene rearrangements or deletions (Dehainault et al. Nucleic Acid Res. 2004 32(18):pe139).

### SUMMARY OF THE INVENTION

An object of the present invention is to provide methods for detection of aneuploidy.

In these methods, the selected chromosomal targets are amplified by multiplex polymerase chain reaction (mPCR). The mPCR is terminated within the exponential phase of the amplification to provide accurate gene dosage relationships which correlates to the number of chromosomes present. The resulting products are then separated and, if necessary, the product signal is enhanced by post-column fluorescence-high performance liquid chromatography (PCF-HPLC). Data generated can then be normalized to a control amplicon to correct for any amplification variability between samples.

Another object of the present invention is to provide kits for detecting aneuploidy via mPCR followed by PCF-HPLC.

### BRIEF DESCRIPTION OF THE FIGURES

FIG. 1 shows exemplary PCF-HPLC chromatograms of multiple sex-linked chromosomal aneuploidies generated in accordance with the method of the present invention. Once the injections are normalized to reduce amplification variations using the control amplicon (housekeeping gene), the gene dosage relationships can be determined. In this exemplary embodiment, a normal female has 2 copies of the X chromosome and 0 copies of the Y chromosome (a 2:0 ratio, determined by peak height and verified by peak area, trace 1). Aneuploidies shown involve XXX (3:0, trace 2), XXY (2:1, trace 3), and X (1:0, trace 4).

### DETAILED DESCRIPTION OF THE INVENTION

It is desirable to be able to detect aneuploidy using the DNA amplification approaches without the need for polymorphic markers, specially-designed fluorescent probes, or highly trained technicians to analyze the data.

The present invention provides methods for determining aneuploidy of selected chromosomes by multiplex polymerase chain reaction (PCR) and post-column fluorescence high performance liquid chromatography (PCF-HPLC). In these methods aneuploidy is detected by the amplification of selected chromosomal target and control DNA sequences by multiplex PCR, followed by separation of these products and enhancement of the product signal by PCF-HPLC, and normalization of the data to a control amplicon to correct for any residual amplification variability.

The present invention thus provides a rapid method (less than 4 hours for sample preparation, mPCR, and analysis) for accurately determining aneuploidy of selected chromosomes by multiplex polymerase chain reaction (mPCR) and high sensitivity post-column fluorescence detection after separation by non-denaturing high performance liquid chromatography (PCF-HPLC). The use of multiplex PCR coupled with PCF-HPLC allows for analysis of chromosomal aneuploidy in biological samples with limited source DNA (e.g. amniocentesis samples) on an automated platform without the use of fluorescent probes.

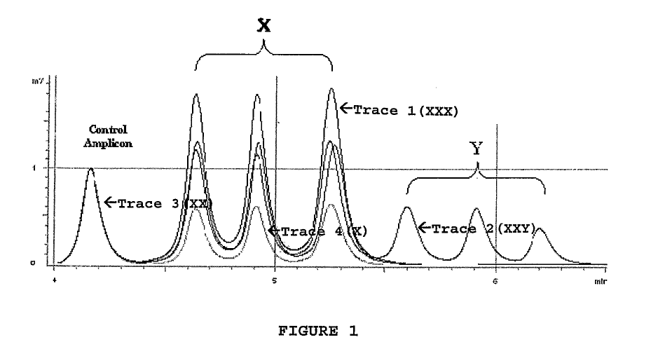

In one embodiment of the invention, a cell sample was assayed for sex chromosome copy number by amplifying three regions on the X chromosome as well as three regions of the Y chromosome. The relative amounts of the amplified targets were then determined after separation by PCF-HPLC and normalization to a control amplicon (located on chromosome 12). This allows for determination of sex (male—1 copy, female—2 copies of the X chromosome; male—1 copy, female—0 copies of the Y chromosome), or diagnosis of a sex chromosome aneuploidy (e.g. XXY, XXX, XYY). See FIG. 1.

Accordingly, the present invention provides a useful method for detecting aneuploidy. While the above example relates to aneuploidies on the X and Y chromosomes, as will be understood by the skilled artisan upon reading this disclosure, the method of the present invention is useful in detecting aneuploidies on any selected chromosomal target.

Important features of the method of the present invention include, but are not limited to, adaptability to detection of aneuploidy in any selected chromosome without using short-tandem repeat (STR) or single-nucleotide polymorphism (SNP) markers or specially-designed fluorescent probes. The use of multiplex PCR coupled with PCF-HPLC allows for analysis of chromosomal aneuploidy in biological samples with limited source DNA (e.g. amniocentesis samples) on an automated platform without the use of fluorescent probes, but within the exponential phase of the PCR reaction to minimize the effect of amplification variability.

The present invention also provides kits for detecting aneuploidy of a selected chromosomal target or targets via mPCR followed by PCF-HPLC. Kits of the present invention comprise a primer pair or primer pairs for amplification via mPCR of a selected chromosomal target or selected chromosomal targets. In a preferred embodiment, the kits of the present invention further comprise a control DNA sequence and or a control amplicon to correct for any residual amplification variability, as well as polymerase, polymerase buffer, dNTPs, directions relating to associated conditions for amplification using a thermal cycler and data analysis, and/or software for automated analysis.

The following nonlimiting examples are provided to further illustrate the present invention.

### EXAMPLES

### Example 1

### Detection of Multiple Sex-linked Chromosomal Aneuploidies

A multiplex PCR was designed to detect aneuploidies of X and Y chromosome. The reaction included a control amplicon which is not located on a sex-linked chromosome (GAPDH gene from chromosome 12), three amplicons from genes located on the X chromosome (HPRT, MTM1 and L1CAM), and three amplicons from genes located on the Y chromosome (SRY, UTY and SMCY genes). Table 1 shows the Genbank Accession numbers as well as primer sets used for amplification.

TABLE 1

Genes used for aneuploidy analysis along with the
associated Genbank Accession Numbers

| | | | | |
|---|---|---|---|---|
| | | Genbank | | |
| | | Accession | | |
| Chromosome | Gene | Number | Primer Sequence | SEQ ID NO: |

| | | | | | |
|---|---|---|---|---|---|
| 12 | GAPDH | J04038 | F: | gtgactaaccctgcgctcct | 1 |
| | | | R: | acccacttctttgatttaccaga | 2 |
| X | HPRT | M26434 | F: | agttgagtttggaaacatctgga | 3 |
| | | | R: | ttaggaatgcagcaactgaca | 4 |
| X | MTM1 | AF020668 | F: | aggttcgctttgaaacagga | 5 |
| | | | R: | aaagaggatcccagtttgcag | 6 |
| X | L1CAM | M74387 | F: | tcagaggttccagggcatc | 7 |
| | | | R: | tcctcgccacgagacaact | 8 |
| Y | SRY | L10101 | F: | ctcttccttcctttgcactga | 9 |
| | | | R: | tatcccagctgcttgctgat | 10 |
| Y | UTY | NM_007125 | F: | atcaccgaaggcaacagc | 11 |
| | | | R: | gcttcggtagcttaagtctttgc | 12 |
| Y | SMCY | NM_004653 | F: | tgggccttatggctaaggat | 13 |
| | | | R: | acgttcccacctccacttt | 14 |

The multiplex PCR was performed in a 0.2 ml PCR tubes with a total volume of 25 μl containing 12.5 μl 2× PCR Master Mix (including dNTPs, buffer, MgCl₂ and Taq DNA polymerase, Promega, Madison, Wis., USA), 50 ng of genomic DNA, and primers which ranged in concentration from 0.2 μM to 1.0 μM. The final volume was adjusted to 25 μl with water. For amplification, an initial 2 minute denaturation at 95° C. was followed by 14 touchdown cycles of 94° C. for 30 seconds, 59° C. for 30 seconds (reduced by 0.5° C. per cycles for 14 cycles to 52° C.), and 72° C. for 45 seconds, 8 cycles of 94° C. for 30 seconds, 52° C. for 30 seconds and 72° C. for 45 seconds, and a final extension at 72° C. for 5 minutes. PCR was carried out in an Applied Biosystems 9700 thermal cycler (Perkin-Elmer, Boston, Mass., USA).

The resulting PCF-HPLC chromatogram is shown in FIG. 1. Gradient conditions used to generate this chromatogram are shown in Table 2.

TABLE 2
Gradient (flow rate: 0.900 ml/minute, analysis temperature:
50.0° C., total run time: 12.7 minutes).

| Time | | | |
|---|---|---|---|
| (minutes) | % A | % B | % D |
| 0.0 | 55.0 | 45.0 | 0.0 |
| 0.5 | 50.2 | 49.8 | 0.0 |
| 2.5 | 44.8 | 55.2 | 0.0 |
| 4.4 | 41.6 | 58.4 | 0.0 |
| 6.4 | 39.5 | 60.5 | 0.0 |
| 8.3 | 38.0 | 32.0 | 0.0 |
| 8.4 | 0.0 | 0.0 | 100.0 |
| 8.9 | 0.0 | 0.0 | 100.0 |
| 9.0 | 55.0 | 45.0 | 0.0 |
| 9.9 | 55.0 | 45.0 | 0.0 |

Data was normalized using the housekeeping gene, GAPDH, to reduce amplification variations. As shown in FIG. 1, a normal female has 2 copies of the X chromosome and 0 copies of the Y chromosome (a 2:0 ratio, determined by peak height and verified by peak area, trace 1). Aneuploidies shown involve XXX (3:0, trace 2), XXY (2:1, trace 3), and X (1:0, trace 4).

What is claimed is:

1. A method for detecting aneuploidy comprising:
amplifying selected chromosomal targets in a biological sample containing DNA by multiplex polymerase chain reaction (mPCR);
terminating the mPCR within an exponential phase of amplification;
separating and enhancing a signal of resulting products by post-column fluorescence-high performance liquid chromatography (PCF-HPLC); and
normalizing data generated from the signal of the separated resulting products to a control amplicon to correct for any residual amplification variability so that aneuploidy is detected.

2. The method of claim 1 wherein the biological sample contains limited source DNA.

3. The method of claim 2 wherein the biological sample is an amniocentesis sample.

4. The method of claim 1 wherein said method is performed on an automated platform.

5. A kit for detecting aneuploidy in a selected chromosomal target or selected chromosomal targets via mPCR followed by PCF-HPLC.

6. The kit of claim 5 comprising a primer pair or primer pairs for amplification of the selected chromosomal target via mPCR and a control amplicon to correct for any residual amplification variability.

7. The kit of claim 6 further comprising polymerase, polymerase buffer, dNTPs, directions relating to associated conditions for amplification using a thermal cycler and data analysis or software for automated analysis.
